(19) United States
(12) Patent Application Publication
(10) Pub. No.: US 2007/0238979 A1
(43) Pub. Date: Oct. 11, 2007

(54) Reference Devices for Placement in Heart Structures for Visualization During Heart Valve Procedures

(75) Inventors: Rany Huynh, Charlestown, MA (US); Nasser Rafiee, Andover, MA (US); Alex Hill, Minneapolis, MN (US); Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/689,961

(22) Filed: Mar. 22, 2007

Related U.S. Application Data
(60) Provisional application No. 60/743,687, filed on Mar. 23, 2006.

(51) Int. Cl. A61B 5/05

(52) U.S. Cl. 600/420; 600/407

(57) ABSTRACT

Visualization reference devices to aid in non-direct visualization of heart structure. The devices are positionable in the heat structure and visible with a desired imaging modality. The devices being elastically transformable between delivery configurations and deployment configurations. The devices being used to assist a clinician in mapping the heart structure while implanting therapeutic devices therein. An example would be using the devices disclosed herein to map the size, location, orientation and displacement of a mitral valve annulus for catheter based implantation of a valve repair device.

### CROSS-REFERENCE TO RELATED APPLICATION

The present application claims priority to U.S. Provisional Application No. 60/743,687, filed Mar. 23, 2006 and titled “Reference Devices for Placement in Heart Structures for Visualization During Heart Valve Procedures”, the entire contents of which is incorporated herein by reference in its entirety.

### TECHNICAL FIELD

This invention relates generally to medical devices and particularly to a device, system, and method for aiding implantation of a heart valve repair device.

### BACKGROUND OF THE INVENTION

Heart valves, such as the mitral and tricuspid valves, are sometimes damaged by diseases or by aging, which can cause problems with the proper function of the valve. The mitral and tricuspid valves consist of leaflets attached to a fibrous ring or annulus. In a healthy heart, the mitral valve leaflets overlap during contraction of the left ventricle, or systole, and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

A common repair procedure involves implanting an annuloplasty ring on the superior, or atrial, surface of the mitral valve annulus. The annuloplasty ring is aligned with the valve annulus and then fixedly attached to the valve annulus. The annuloplasty ring generally has a smaller diameter than the distended valve annulus, and when attached to the annulus, the annuloplasty ring draws the annulus into a smaller configuration, bringing the mitral valve leaflets closer together and providing improved valve closure during systole.

Catheter-based repair procedures for implanting devices on the valve annulus require non-direct visualization of, at least, the heart valve and the device during placement on the valve annulus. As used herein, the phrase non-direct visualization refers to viewing an indirect image of body tissues and/or devices within a patient using some method other than direct visualization without the aid of any device. Non-direct visualization of the valve annulus is challenging.

Cardiac tissues do not appear under fluoroscopy, making it very difficult to accurately align the valve repair device prior to its implantation. In many procedures, radiopaque contrast dye is used with x-ray imaging equipment to increase the visualization of the area of interest. However, when treating the mitral valve, repeated injections of contrast dye are not practical because of rapid wash-out in the high-flow area being treated. Additionally, to make the high-volume contrast injections, the annuloplasty catheter system would require more lumens, larger lumens, or an additional catheter, none of which is desirable during catheterization procedures. Furthermore, multiple high-volume contrast injections are not desirable for the patient due to potential complications in the renal system, where the radiopaque contrast medium is filtered from the blood.

Other techniques for viewing images of heart structures include ultrasonography such as trans-thoracic echocardiography (TTE), trans-esophageal echocardiography (TEE), and cardiac magnetic resonance (CMR) including magnetic resonance imaging (MRI) or magnetic resonance angiography (MRA). However, none of the above techniques, used alone or in combination provides adequate visualization and guidance during catheter-based valve repair procedures.

Annuloplasty procedures are further challenged by the structure of the valve annulus and the fact that the annulus can undergo significant movement during procedures performed on a beating heart. In particular, the mitral valve annulus lacks a definable shelf or ledge for conveniently locating an implantable valve repair device. The mitral valve leaflets are little more than flaps or appurtenances attached to the cardiac muscle tissue, creating a pseudo-annulus. During systole, the mitral valve is closed to form a relatively flat floor of the left atrium. However, during diastole, the mitral valve leaflets open towards the ventricular walls such that, in many cases, the valve annulus is not well defined. Since annuloplasty is performed on a beating heart, care must be taken during both systole and diastole when positioning an implantable valve repair device for fixation.

Without the direct optical visualization that is possible during surgery, it is difficult to position an implantable device in abutment with the superior surface of the valve annulus. With non-direct imaging techniques used during a catheter-based procedure, an implantable valve repair device may be inadvertently affixed in a misaligned position below, above or angled across the valve annulus. Affixing the implantable valve repair device in such a misaligned position could have negative consequences for the patient, such as increasing mitral regurgitation and/or triggering ectopic heart beats.

Therefore, it would be desirable to provide a device, system, and method for aiding implantation of a valve repair device to overcome the aforementioned and other disadvantages.

### SUMMARY OF THE INVENTION

The present invention provides visualization reference devices for placing in the heart structure to aid in visualization of the heart during therapeutic procedures. An example of such procedures would be repair of cardiac valves. A specific example would be catheter-based or other minimally invasive valve repair device implantation to treat mitral regurgitation. The structures may be temporarily disposed in the heart structure and comprise imageable material to aid non-direct visualization of the valve annulus. The shape and size if the devices are selected based on the location in the heart structure where the device will be implanted. The devices can be elastically recoverable from the heart structure and they can be delivered via catheter in one configuration, assume a second configuration during the procedure, and then be reconfigured for recovery from the heart structure. The visualization reference devices may also include inflatable portions that can be temporarily expanded in the heart structure.

As used throughout this document, the term “visualize” means to make visible on a display device while using some visualization technique that is used for medical procedures. Thus a visualization reference device is a device that will be visible to a treating clinician during a cardiac procedure and provide the clinician with information about the portion of the anatomy where the visualization reference device is located. Additionally, as used herein the terms heart structure means the veins, arteries, chambers, and valves located on, in, or in proximity to a heart.

One object of the current invention is to provide devices that can be used to assist in the non optical visualization of areas of minimal motion in the heart structure, such as the aortic valve. This can be important for catheter based mitral valve annuloplasty because the aortic cusp shares the same wall as the anterior leaflet, but does not experience the motion that the mitral valve does.

Another object of the current invention is to provide devices that can be used to assist in the non optical visualization of the heart structure such that the location of the mitral valve annulus can be identified or predicted. Examples of such structure would be the coronary sinus, the left circumflex artery, and other vessels in the coronary vasculature.

Yet another object of the current invention is to provide methods that can be used to assist in the non optical visualization of the posterior commisure of a mitral valve. For example marking the location of the fossa ovalis would provide assistance in location the posterior commisure, and thus provide assistance in determining the location of the mitral valve annulus.

It is also an object of the current invention to provide devices that can be used to assist in determining the motion of the beating heart. This may be accomplished by placing a catheter in the heart structure such that the distal tip of the catheter rests on the mitral valve annulus. The tip can then be observed from dyastole to systole and measurements can be made to determine movement of the heart structure during the heart beat cycle. Catheters and other devices can also be placed in other heart structure to determine movement.

One aspect of the present invention provides for placing a visualization reference device in the aortic valve. The device can be a wire designed to form a coil inside of the aortic valve area or it can be a woven device designed to expand within the aorta upon expulsion from a delivery catheter.

Another aspect of the invention provides for placing a visualization reference device in the fossa ovalis. Another aspect of the invention provides for placing a visualization reference device, in the form of a catheter, into the left atrium of a heart and resting it on the mitral valve annulus.

Another aspect of the invention provides a wire with markers spaced along the wire at known intervals. The wire can be inserted into the coronary sinus or other coronary vasculature. One aspect of the invention provides for a marker wire placed in the coronary sinus and another wire placed in the intraventricular vein.

The visualization reference devices of the current invention can be made completely or in part from material having a desired degree of visibility when using non-direct visualization technology. Alternately, the devices can be completely or partially coated with materials that are visible when using non-direct visualization, or they can include coils having electro magnetic or electro potential properties. When using a visualization reference device, alone or in a combination with other devices, a clinician can take multiple images of a heart in dyastole and systole. These images can be taken from different angles or the same angle and they can be superimposed upon each other to provide a clinician with a good image of the heart structure and the location of a valve annulus.

Another aspect of the present invention is a system for aiding procedures such as catheter-based or other minimally invasive valve repair device implantation. The system comprises a delivery catheter including a lumen with an exit port, and a visualization reference device having a delivery configuration that is slidably positionable within the lumen. The visualization reference device is deployed through the exit port when the delivery catheter has been navigated to a target site. Upon exiting the catheter, the visualization reference device assumes a pre-formed deployment configuration. A clinician performing a procedure uses the device, alone or in combination with other devices in other locations, to assist in locating the annulus and placing a device for treating heart valve regurgitation.

Yet another aspect of the present invention is a method of treating a cardiac valve. The method comprises delivering a visualization reference device in a delivery configuration to a location in the heart structure via a delivery catheter, deploying the visualization reference device through an exit port of the delivery catheter, positioning the visualization reference device in the desired location in the heart structure and non-directly viewing at least a marked portion of the positioned reference ring.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIGS. 1 and 2 are illustrations of reference wires having spaced visualization portions, in accordance with the present invention.

FIG. 3A illustrates a diaphragmatic aspect of a heart.

FIG. 3B illustrates a sternocostal aspect of the heart of FIG. 3A.

FIG. 4 illustrates a top view of a heart.

FIGS. 5 and 6 are illustrations showing the placement of reference wires in accordance with the current invention.

FIGS. 7 and 8 are illustrations of visualization reference devices for placement in the aorta, in accordance with the current invention.

FIG. 9 is an illustration showing a catheter used as a locating marker, in accordance with the current invention.

FIG. 10 is a flow diagram of one method of using the visualization reference devices to map the location of a cardiac valve annulus, in accordance with the current invention.

### DETAILED DESCRIPTION

The invention will be describe by reference to the drawing figures, where like numbers refer to like parts. One aspect of the present invention is to provide visualization reference devices that have non-direct visualizable properties, for aiding in placement of an implantable valve repair device in abutment with a heart valve annulus. Documents disclosing devices for valve repair are U.S. Patent Application having the Publication No. 2007/005,1377, entitled “Cardiac Valve Annulus Reduction System,” by Douk et al. and U.S. Patent Application having the Publication No. 2007/002,7533, entitled “Cardiac Valve Annulus Restraining Device,” by Douk, the contents of these applications is incorporated herein by reference thereto.

The visualization reference devices are designed to be temporarily positioned using intravascular catheterization techniques. Alternatively, surgical or minimally invasive, i.e. endoscopic techniques may be used to place the devices.

The reference devices of the current invention can be viewed using ultrasonography, echocardiography, trans-thoracic echocardiography (TTE), trans-esophageal echocardiography (TEE), and cardiac magnetic resonance (CMR) including magnetic resonance imaging (MRI), or magnetic resonance angiography (MRA). Use of the devices with those imaging techniques provides better visualization of the heart structure that use of these techniques without the visualization reference devices.

The terms “distal” and “proximal” are used herein with reference to the treating clinician during deployment of the device; “Distal” indicates an apparatus portion distant from, or a direction away from the clinician and “proximal” indicates an apparatus portion near to, or a direction towards the clinician. The reference devices of the current invention may be made, in whole or in part, from one or more materials that are viewable by radiography, ultrasound, or magnetic resonance imaging visualization techniques. Embodiments of the devices may also be coated with materials that are visible using such visualization methods.

The discussion below relates to placement of visualization reference devices in heart structure for use during mitral valve repair procedures. However, those with skill in the art will recognize that visualization reference devices of the invention may also be deployed at other cardiac valves or other locations in the body and may be used to visualize other openings or other structures within the body.

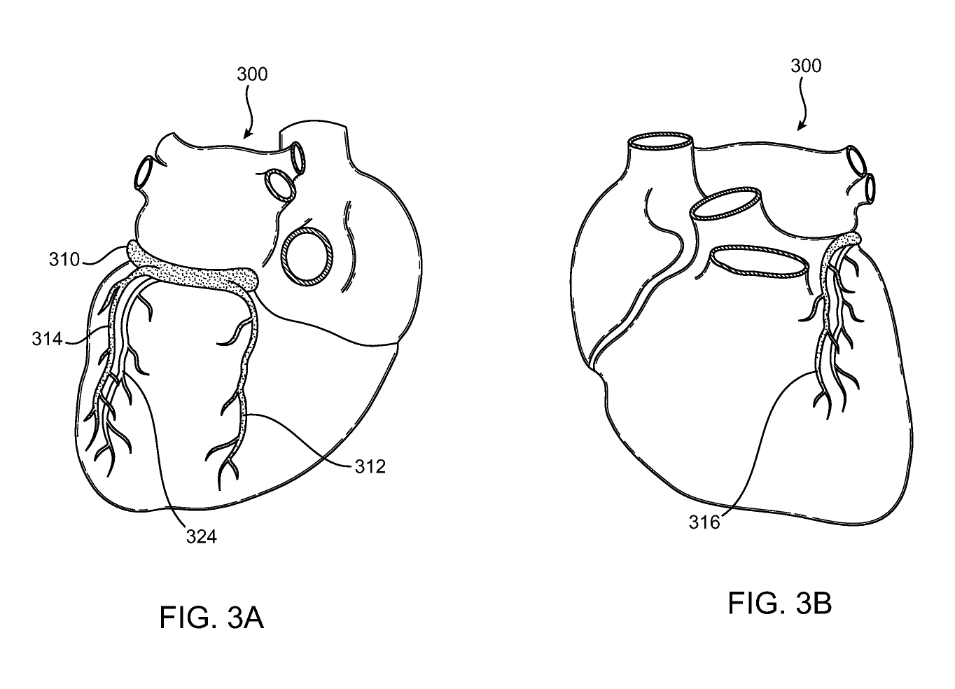

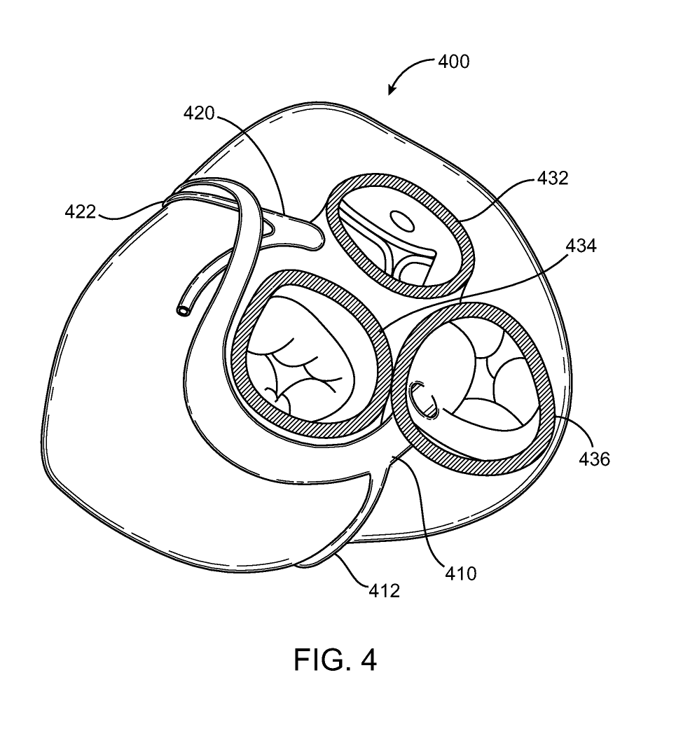

Referring to FIG. 3A, there can be seen an illustration of the diaphragmatic aspect of a heart 300 showing the coronary sinus 310 with the middle cardiac vein 312 and the posterior vein of the left ventricle 314 branching from the coronary sinus. Also shown in FIG. 3A is the ventricular branch 324 of the coronary artery. FIG. 3B illustrates a sternocostal aspect of the heart of FIG. 3A showing the left marginal vein 316 descending from the coronary sinus and the anterior descending branch of the coronary artery. FIG. 4 is an illustration showing a heart 400 as seen from above showing the aorta 432 and the left coronary artery 420. Also shown in the figure are the left atrium 434 and the right atrium 436. The coronary sinus 410 enters the right atrium above the valve. It should be noted that FIGS. 3A, 3B, and 4 are shown to illustrate the general location of heart structure and they are not intended to be exactly anatomically correct.

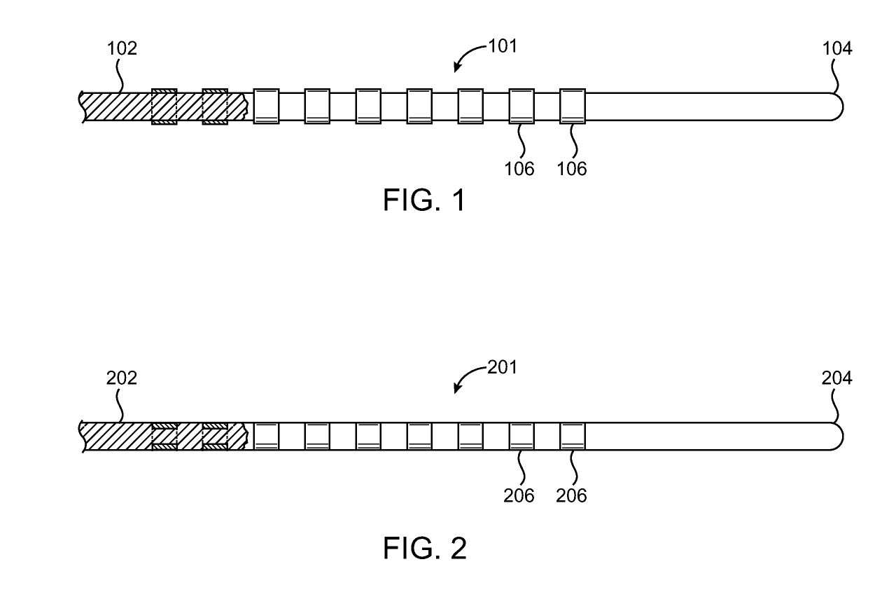

FIG. 1 illustrates a device for aiding in non-direct visualization of a valve annulus during an annuloplasty procedure. The device 101 can be delivered to a location within a heart structure via a delivery catheter (not pictured) or it can be navigated through the vasculature similar to the way that a guidewire is navigated. The device tip 104 may be a straight tip or a preformed or deflectable distal tip that is capable of assuming a desired bend with respect to the longitudinal axis of the device to aid in delivering a visualization reference device.

In one embodiment of the invention, the device may be delivered in a delivery catheter having a preset curve, e.g., a pigtail-shaped tip as such curves are known in the catheter art. Pigtail-shaped catheter tips are known to facilitate crossing an aortic valve and to minimize ectopic heartbeats when located within the left ventricle.

The reference wire device 101 has a wire section 102 that is similar in construction to a guidewire used for catheterization procedures. The wire section will extend outside of the body of a patient and be controllable by a clinician who has access to the proximal end of the reference wire. The reference wire 101 includes a plurality of marker portions 106 that are visible using one of the above noted visualization techniques or other non-direct visualization methods known in the art. The marker portions are evenly spaced at a predetermined distance along the reference wire. The marker portions of the device depicted in FIG. 1 have a diameter that is slightly bigger than the diameter of the reference device. In at least one embodiment, the marker portions are coated with a material containing bismuth.

The marker portions of the device can be made completely from a material that is visible using a desired visualization technique, or they can be coated by such material. In at least one embodiment, the marker portions

A distal tip portion 124 is located at the end of the reference wire. The distal tip portion may be a tight helically wound. In one embodiment, the reference wire may be made from stainless steal, the marker portions and the tip portion are at least platinum coated, and the reference wire between the marker sections is also helically coiled such that it forms a flexible “spring section.”

FIG. 2 illustrates an embodiment 201 of a reference wire according to the current invention wherein the reference wire has a wire section 202, and a distal tip portion 224. is located at the end of the reference wire. The marker portions are evenly spaced along the reference wire and have the same diameter as the diameter of the reference device.

The reference wires of the current invention can be placed within the vascular structure of a heart, or they can be placed within the hearts chambers. Visualization of the wires will allow a clinician to have some reference points for use when performing catheter based procedures inside of a beating or temporarily stopped heart. Having evenly spaced marker portions where the distance between the portions is know, can allow a clinician to select an appropriately sized device for use in repairing heart valves.

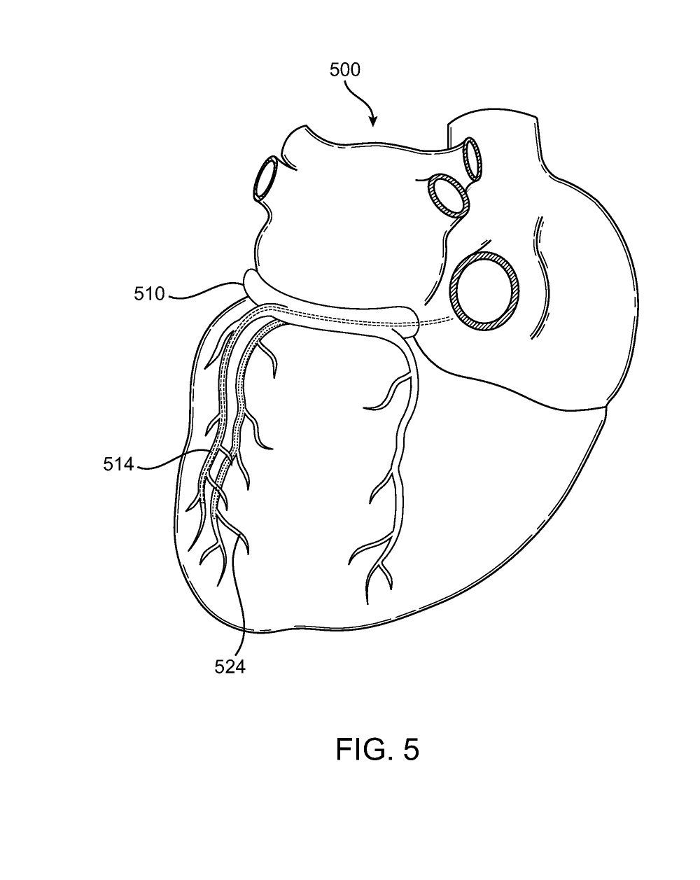

FIG. 5 shows an illustration of a heart 500 having a reference wire (represented by the double dotted lines) extending into the ventricular branch 524 of the coronary artery. Another reference wire is extended through the coronary sinus 510 and into the posterior vein of the left ventricle 514. The reference wire in the coronary artery can be delivered through the vasculature using the same routes known in the art for coronary angioplasty or other coronary procedures. The reference wires of the current invention can be placed into the coronary sinus by inserting the device into the femoral vein, navigating to the inferior vena cava into the right atrium and then into the coronary sinus.

Placing the reference wire in the coronary sinus gives a treating clinician information she or he can use to determine the location and planar orientation of a mitral valve annulus. This is because, in most cases, the coronary sinus runs parallel to the mitral valve annulus for about 75% of the circumference of the valve and it is generally even with the annulus.

Placing a reference wire in the ventricular branch of the coronary artery or the posterior vein of the left ventricle gives a clinician information he or she can use to determine the location of the commissures of the mitral valve and/or the papillary muscles in the left ventricle.

Placing reference wires in both the coronary artery and the coronary sinus will allow a clinician to get a fuller appreciation of the mitral valve location than a single wire in either vessel would allow.

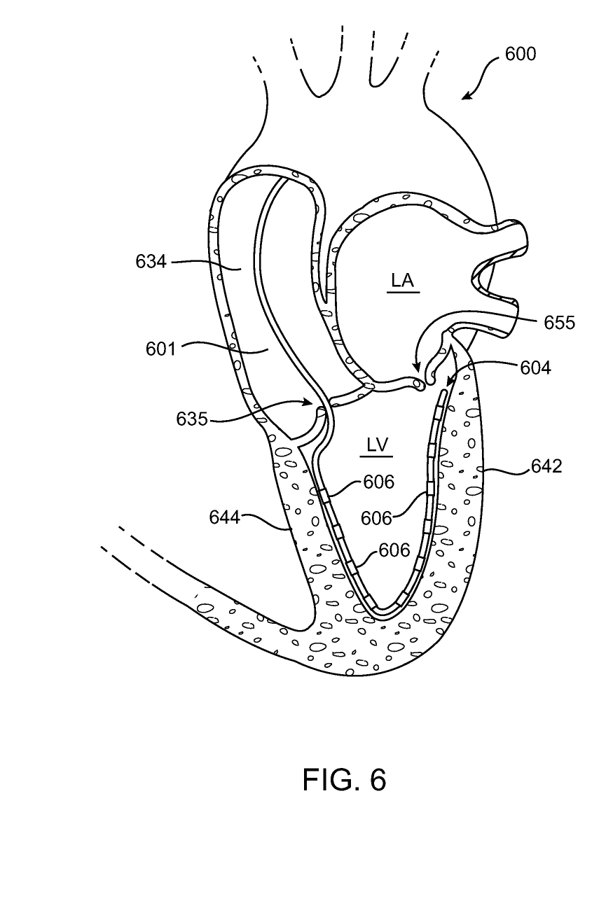

FIG. 6. is an illustration showing a left atrium LA and left ventricle LV of a heart 600. A reference wire 601 having a tip 604 and a plurality of marker portions 606. The wire extends from the aorta 634 and through the aortic valve 635. The reference wire is positioned in the left ventricle and shaped such that it rests against the septum 644 and the free wall 642 of the ventricle. Placing a wire in the ventricle allows a clinician to visualize the shape and size of the ventricle so that he or she can determine the location of the mitral valve 655.

Visualization reference wires of the current invention can be placed just about anywhere in the heart structure where they would provide some advantage in helping to determine the shape and size of a heart. Additionally, adding specifically spaced apart visualization portions allows a clinician to determine the size of the heart and thus the size of a treatment device for addressing valvular disease. For instance, a clinician can tell that a coronary sinus wraps along the valve annulus for a determined number of spaced apart portions and subsequently be able to correlate that distance to a length for the coronary sinus. Knowing the length of the coronary sinus and using that knowledge in combination with information gathered from other visualization reference devices, may enable a clinician to determine an optimum size for a treatment device. The information will also allow a clinician to better visualize a procedure while placing a treatment device in a heart. Once the procedure is completed or the reference wire is no longer needed, it can be withdrawn from the body with relative ease. The reference wire of at least one embodiment of the invention can be visualized while it is still in a catheter, so it does not have to be expelled from a delivery catheter during a heart valve procedure.

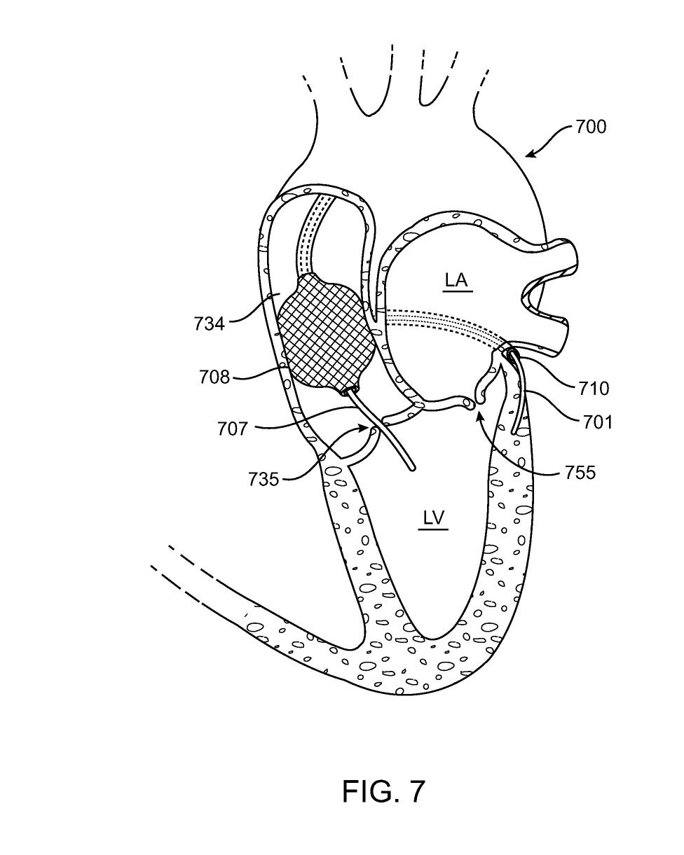

Referring now to FIG. 7 there can be seen an illustration of a visualization reference device 708 placed adjacent an aortic valve 735 of a heart 700 in combination with a reference wire 701 that is placed in the coronary sinus 710. As can be seen in the figure, the aorta locating device 708 is located in the aorta 734 directly above the aortic valve, and it is delivered through the vasculature by catheter. The depicted device has a centering wire 707 that is placed through the aortic valve 735 to help center the device in the aorta. Placing the device in the aorta allows a clinician to locate the aortic cusp, which shares a wall with the anterior leaflet of the mitral valve 755.

The aorta reference/locator device can be made from a tubular braid of material having the desired visualization properties. The aorta reference device can be made to be self expanding, balloon expandable or expandable by some mechanical action. The aorta locator device can be constructed from a biocompatible material having suitable visualization properties to assist a clinician in non-direct visualization of the heart structure. In one embodiment, the device is made from nitinol.

In a delivery configuration, the tubular, braided reference devices have a relatively small outer diameter to allow them to pass through a delivery catheter or other delivery member. Once the tubular, braided reference devices are deployed, they can self expand or be manipulated to assume a deployment configuration where at least a portion of the tubular braid expands radially outward such that the deployed device can be easily viewed using the desired imaging modality. In one embodiment, the aorta visualization reference device can be expanded such that it will brace against the walls of the aorta, where it will be secured until the procedure is completed.

The tubular, braided reference devices can be configured such that they can be collapsed after the procedure is completed, so that the device can be withdrawn into a delivery catheter and then removed from the body. The device can be configured such that it can be collapsed after some mechanical manipulation or the aorta reference device can be self collapsing. The self collapsing devices may require some manipulation to open and remain in the open position, but the device will then collapse once when any opening force is removed.

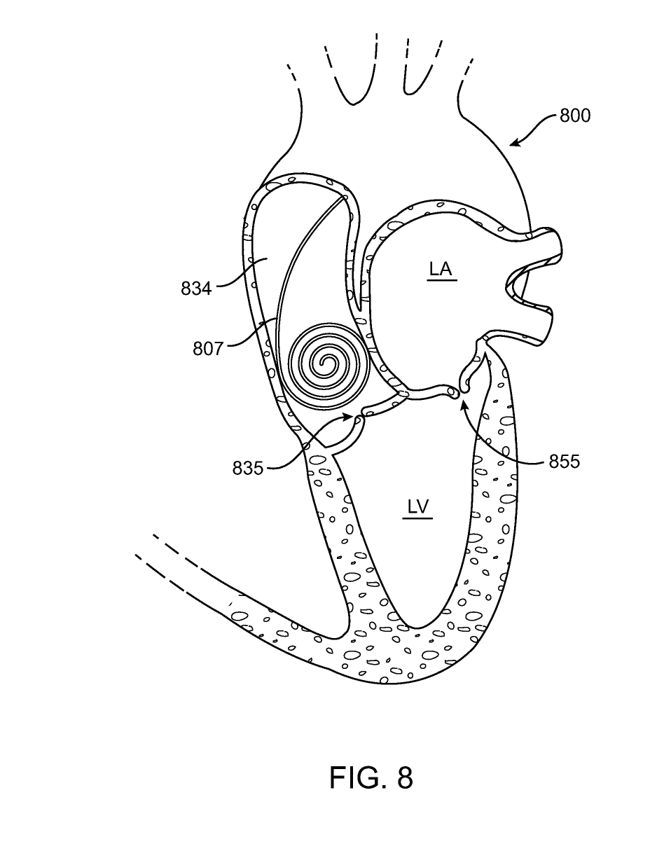

FIG. 8 shows an alternate embodiment of a visualization reference device 707 for placement in an aorta 834. The device 707 is a shaped wire having the desired visualization properties based on the imaging modality that is being used. The device can be made from some biocompatible shape memory material and it can be pre-formed into any desired shape for placement adjacent an aortic valve 835 of a heart such that it would help a clinician locate and identify the aortic cusp and ultimately the mitral valve 855. Once the procedure is complete, the wire can be withdrawn into the catheter and the catheter can be removed from the patient's body.

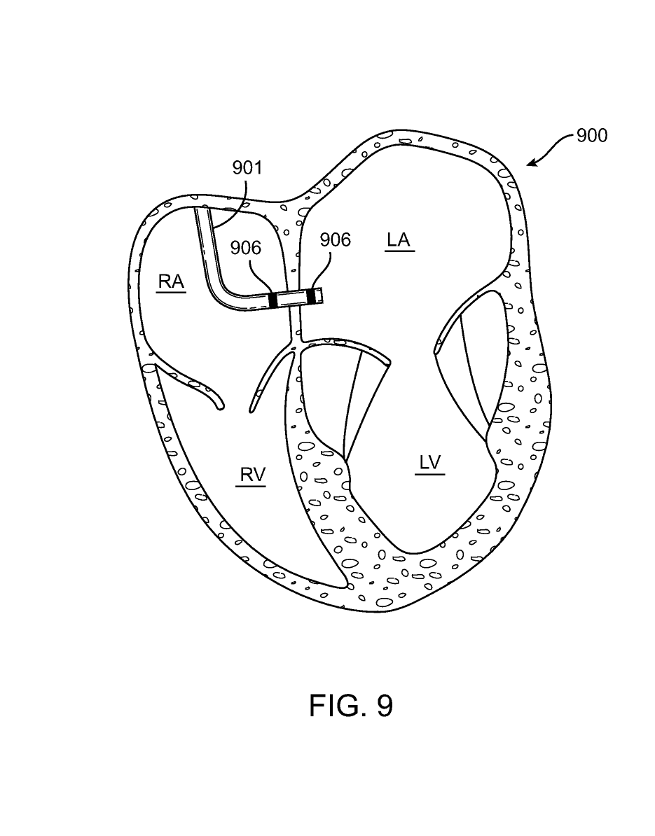

FIG. 9 shows a catheter 901 inserted through the septum separating the right atrium RA and left atrium LA of a heart 900. The catheter 901 can include or be made from materials that are visible using the desired imaging modality, and it can also be used to inject dyes or other contrast agents into the heart. In at least one embodiment, the catheter is used to deliver a device for treating mitral regurgitation. In at least one embodiment of the current invention, the catheter can be placed such that it rests on the mitral valve annulus whereby visualizing and measuring the movement of the catheter during systole and dyastole will give a clinician an idea of how far the mitral valve will be displaced while a device is being implanted.

Also referring to FIG. 9, the location of the fossa ovalis can be marked by placing the catheter through the septum at that location or by injecting a contrast agent at that location.

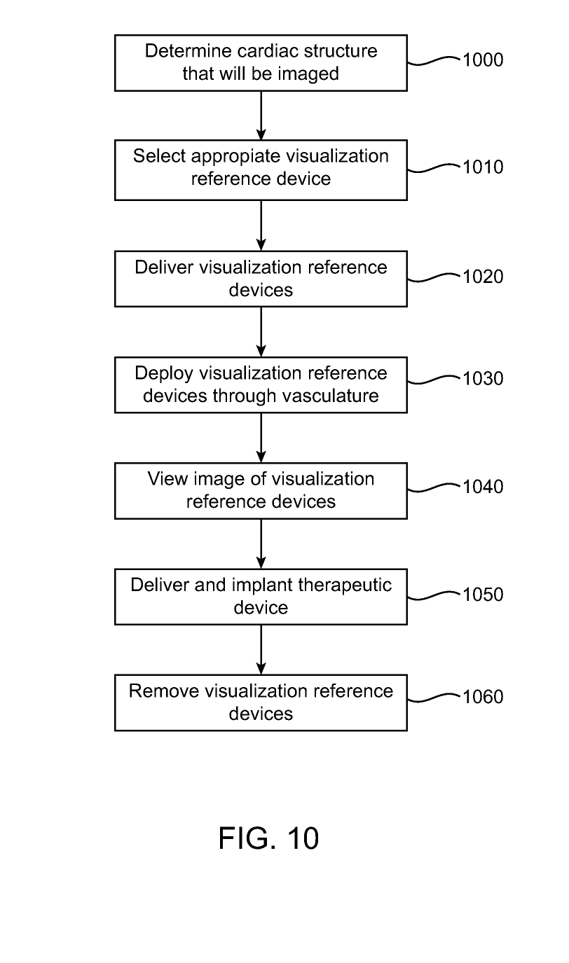

Referring to FIG. 10, to use the visualization reference devices of the current invention, a clinician must determine which imaging modality will be used and what heart structure would be advantageous to visualize during a medical procedure 1000. Appropriate devices are then selected based on imaging modality 1010 and the heart structure to be viewed. The visualization reference devices are delivered to the appropriate locations 1020 and then deployed 1030. Images of the visualization reference devices are viewed 1040 so that the clinician can identify the appropriate location in the heart structure for the implantation of a therapeutic device. A therapeutic device is delivered implanted 1050 and the visualization reference devices are recovered 1060.

The delivery catheters being used to deliver visualization reference devices to the aortic valve, the left ventricle, or the cardiac arteries can be navigated through a patient's circulatory system to the desired location. This may be accomplished by inserting the devices into and through the femoral artery into the aorta. The catheter can then be inserted into the desired artery, delivered to the aortic valve, or be inserted through the aortic valve and into the left ventricle. Catheters may used to deliver the devices or for the procedures described herein, and those catheters may include radiopaque markers or other markers as is known in the art. For minimally invasive techniques or surgical approaches with an open chest, the delivery catheter may be replaced by an elongate element such as an endoscope, trocar or cannula, which may be inserted directly into the ascending aorta. The elongate element can then follow the same path as the catheter-based procedure to reach the desired location.

For visualization reference devices that are being placed in cardiac veins, the coronary sinus, or into the right side of the heart, the devices can be is inserted into either the jugular vein or the subclavian vein, and passed through the superior vena cava and onto the desired location. Alternatively, the devices may be inserted into the femoral vein and passed through the common iliac vein and the inferior vena cava and then to the desired location. Catheters may used to deliver the devices or for the procedures described herein, and those catheters may include radiopaque markers or other markers as is known in the art. For minimally invasive techniques or surgical approaches with an open chest, the delivery catheter may be replaced by an elongate element such as an endoscope, trocar or cannula, which may be inserted directly into the ascending aorta. The elongate element can then follow the same path as the catheter-based procedure to reach the desired location.

The devices disclosed and discussed herein may be made from a suitable biocompatible material including a suitable biocompatible shape-memory material or a suitable biocompatible super elastic material. Embodiments of the devices disclosed herein may be made from biocompatible polymers, biocompatible metals, biocompatible alloys, or a combination thereof. Examples of biocompatible polymers include, but are not limited to, polyurethane, polyethylene, polyamide, fluoropolymers such as fluorinated ethylene propylene (FEP) or polytetrafluoroethylene (PTFE), or polyether-block amide (PEBA) co-polymer. Examples of biocompatible metals and metal alloys include, but are not limited to a nickel-titanium alloy, a nickel-cobalt alloy, another cobalt alloy, stainless steel, combinations thereof, and the like.

The reference devices of the current invention may be made, in whole or in part, from one or more materials that are viewable by radiography, ultrasound, or magnetic resonance imaging visualization techniques. Embodiments of the devices may also be coated with materials that are visible using such visualization methods. Some embodiments of the devices comprise a combination of materials that allows for viewing an image of the device using two or more of the above-mentioned techniques.

Some embodiments of the visualization reference devices can include materials having a high X-ray attenuation coefficient (radiopaque materials). The devices may be made in whole or in part from the material, or they may be coated in whole or in part by radiopaque materials. Alloys or plastics may include radiopaque components that are integral to the materials. Examples of suitable radiopaque material include, but are not limited to gold, tungsten, silver, iridium, platinum, barium sulfate and bismuth sub-carbonate.

The visualization devices may include materials having electro magnetic properties or electro potential properties. Such devices would require an energy source exterior to the body of a patient that was being treated by methods using the visualization reference device disclosed herein.

One embodiment of the current invention may include devices coated with an echogenic material, such as closed cell foam, microporous, mezoporous or other porous material. In another embodiment, the device may be made from a polymer having a plurality of embedded micro bubbles or a microporous surface structure, such as ECHO-COAT® medical imaging coating by Angiotech BioCoatings, Inc. of Henrietta, N.Y., U.S.A. The micro pores of such materials are readily visualized using ultrasonographic techniques.

Embodiments of the visualization reference devices may contain one or more MRI-visible components such as ferromagnetic, paramagnetic or diamagnetic particles, or compounds found in liquid MRI contrast agents. These agents include, but are not limited to, ultra small super paramagnetic iron oxide (USPIO), e.g. Combidex® positive contrast agent by Advanced Magnetics, Inc. of Cambridge, Mass. U.S.A., or negative contrast agents such as low-molecular-weight gadolinium chelate, gadolinium tetraazacyclododecanetetraacetic acid (Gd-DOTA) or perfluoroctylbromide (PFOB). Alternatively, the visualization devices may encompass helical and ring structures arranged such that an applied electromagnetic field will induce radiation of an electromagnetic field that improves visualization of a medical device under MRI, as taught in U.S. Pat. No. 6,802,857 entitled MRI Stent.

What is claimed is:

1. A system for aiding in the visualization of heart structure during the implantation of a therapeutic device in side of a heart, the system comprising:
at least one visualization reference device configured for placement within the coronary arteries, coronary sinus, aorta, or a heart chamber for treating valvular disease; and
the at least one device is viewable using radiography, fluoroscopy, ultrasonography, trans-echocardiography cardiac magnetic resonance, or magnetic resonance imaging.

2. The system of claim 1 wherein the at least one visualization reference device comprises a plurality of visualization reference devices.

3. The system of claim 1 wherein the at least one visualization reference device comprises an elongated reference wire having a distal end, a proximal end, and a plurality of marker portions; and the marker portions are viewable using radiography, fluoroscopy, ultrasonography, trans-echocardiography cardiac magnetic resonance, or magnetic resonance imaging.

4. The system of claim 1 wherein the at least one visualization device comprises an expandable element made from a braided material.

5. The system of claim 1 wherein the at least one visualization reference device comprises a biocompatible material chosen from a group consisting of nitinol, stainless steel, age-hardenable nickel-cobalt-chromium-molybdenum alloy, engineering plastic, amides, polyimides, polyolefins, polyesters, urethanes, thermoplastics, thermoset plastics, and blends, laminates or copolymers thereof and combinations thereof.

6. The system of claim 1 wherein at least a portion of the at least one visualization reference device comprises a material viewable by at least one imaging technique selected from a group consisting of radiography, fluoroscopy, ultrasonography, echocardiography, and magnetic resonance imaging.

7. The system of claim 1 wherein at least a portion of the at least one visualization reference device is coated with a material viewable by at least one imaging technique selected from a group consisting of radiography, fluoroscopy, ultrasonography, echocardiography, trans-esophageal echocardiography and magnetic resonance imaging.

8. The system of claim 1 wherein the at least one visualization reference device includes a porous material viewable under ultrasonography.

9. The device of claim 1 wherein the at least one visualization reference device comprises a material chosen from a group consisting of ferromagnetic, paramagnetic and diamagnetic particles, and ultrasmall super-paramagnetic iron oxide, low-molecular-weight gadolinium chelate, gadolinium tetraazacyclododecanetetraacetic acid, and perfluor-octylbromide.

10. A system for aiding in the visualization of heart structure during the implantation of a therapeutic device in side of a heart, the system comprising:
at least one elongated visualization reference wire having a distal end, a proximal end, and a plurality of marker portions evenly spaced along at least a part of the visualization reference wire viewable using radiography, fluoroscopy, ultrasonography, trans-echocardiography cardiac magnetic resonance, or magnetic resonance imaging;
at least one expandable braided visualization reference device viewable using radiography, fluoroscopy, ultrasonography, trans-echocardiography cardiac magnetic resonance, or magnetic resonance imaging; and
at least one catheter for positioning the at least one expandable braided visualization reference device within the structure of a heart.

11. The system of claim 10 wherein the at least one visualization reference wire comprises a plurality of visualization reference wires.

12. The system of claim 10 wherein expandable braided visualization reference device comprises a plurality of expandable braided visualization reference devices.

13. The system of claim 10 wherein the at least one elongated visualization reference wire and the at least one visualization reference device each comprise a biocompatible material chosen from a group consisting of nitinol, stainless steel, age-hardenable nickel-cobalt-chromium-molybdenum alloy, engineering plastic, amides, polyimides, polyolefins, polyesters, urethanes, thermoplastics, thermoset plastics, and blends, laminates or copolymers thereof and combinations thereof.

14. A method for providing visualization references during procedures for heart valve treatment, the method comprising the steps of:
determining heart structure that will be visualized during the procedure;
selecting at least one appropriate visualization reference device for the structure to be visualized;
delivering the at least on visualization reference device to the desired heart structure;
deploying the at least one visualization reference device within the desired heart structure;
viewing the image of the at least one visualization reference device;
delivering and implanting a therapeutic device within the heart; and
removing the at least one visualization reference device from the heart structure.

15. The method of claim 14 wherein the at least one visualization reference device comprises a plurality of visualization reference devices.

16. The method of claim 14 wherein the at least one visualization reference device comprises an elongated reference wire having a distal end, a proximal end, and a plurality of marker portions; and the marker portions are viewable using radiography, fluoroscopy, ultrasonography, trans-echocardiography cardiac magnetic resonance, or magnetic resonance imaging.

17. The method of claim 14 wherein the at least one visualization device comprises an expandable element made from a braided material.

18. The method of claim 14 wherein the at least one visualization reference device comprises a biocompatible material chosen from a group consisting of nitinol, stainless steel, age-hardenable nickel-cobalt-chromium-molybdenum alloy, engineering plastic, amides, polyimides, polyolefins, polyesters, urethanes, thermoplastics, thermoset plastics, and blends, laminates or copolymers thereof and combinations thereof.

19. The method of claim 14 wherein at least a portion of the at least one visualization reference device comprises a material viewable by at least one imaging technique selected from a group consisting of radiography, fluoroscopy, ultrasonography, echocardiography, and magnetic resonance imaging.

20. The method of claim 14 wherein at least a portion of the at least one visualization reference device is coated with a material viewable by at least one imaging technique selected from a group consisting of radiography, fluoroscopy, ultrasonography, echocardiography, trans-esophageal echocardiography and magnetic resonance imaging.
